(19) United States
(12) Patent Application Publication
(10) Pub. No.: US 2019/0316071 A1
(43) Pub. Date: Oct. 17, 2019

(54) PERFUSION DEVICE

(71) Applicant: Hektros S.r.l., Bozen (IT)

(72) Inventors: Daniel HEKL, Innsbruck (AT); Hermann TROGER, Brixen (IT)

(21) Appl. No.: 16/454,231

(22) Filed: Jun. 27, 2019

Related U.S. Application Data
(63) Continuation of application No. PCT/EP2017/084732, filed on Dec. 28, 2017.

(30) Foreign Application Priority Data
Dec. 30, 2016 (AT) A 51195/2016

(51) Int. Cl. C12M 1/00; C12M 1/22; C12M 1/12

(52) U.S. Cl. CPC C12M 29/10; C12M 25/06; C12M 23/10

(57) ABSTRACT

A perfusion device for a cell culture container, particularly a petri dish, includes an inlet opening for supplying nutrient medium, and an outlet opening for discharging unused nutrient medium. A particularly plate-shaped perfusion region dispenses nutrient medium to cell cultures, and is connected to the inlet opening via a supply line and is connected to the outlet opening via a discharge line. The perfusion region can be inserted into the cell culture container and comprises a geometric structure which connects the supply line and the discharge line in the form of an at least partially, preferably entirely spiral-shaped channel, through which nutrient medium can flow. The geometric structure is spaced apart from the floor of the cell culture container in the inserted state.

### BACKGROUND OF THE INVENTION

The invention concerns a perfusion device for a cell culture container, in particular a petri dish, having the features of the classifying portion of claim 1.

A method of investigating the behaviour of cells or microorganisms involves growing cultures in cell culture containers (so-called culture dishes) and then observing the effects of a nutrient medium which is possibly mixed with medicaments or other test substances. The cell culture containers involve dish-shaped containers like for example petri dishes, at least partially plate-shaped containers like for example multi-well plates or also other containers used for the cultivation of cells or microorganisms.

After the cells are sown into the cell culture container the cells begin to multiply until the entire growth surface of the container is covered with cells. In that case the cells with the container, in the case of a static culture (so-called “batch method”) are left at rest in a special incubator which at body temperature constantly holds a vapour-saturated atmosphere of 95% air and 5% carbon dioxide. Thus the cells statically coated with the nutrient medium receive oxygen by passive diffusion across a gap of the cover of the container, which is not disposed thereon in sealed relationship.

The main problem in that respect are the ambient conditions in static cultures, which vary continuously over the entire culture period. Nutrient substances are absorbed by the cells from the freshly coated nutrient medium and given back into the nutrient medium again “metabolised” in chemically modified form. In that respect the metabolically active cells in the first 12 to 24 hours of the nutrient surplus supply require a very great deal of oxygen for the oxidative breakdown of glucose and amino acids, in which case a diffusion-induced oxygen gradient rapidly occurs in the nutrient medium which is millimeter-thick coated. The cells frequently become hypoxic and increasingly adapt their metabolism to anaerobic glycolysis. In that situation glucose no longer becomes CO₂ but is almost only broken down to lactate and eliminated. Usually the nutrients which are contained in the limited volume of coated nutrient medium are completely consumed after 24 to 48 hours while the nutrient medium at the same time in enriched with excreted cell metabolic products. By virtue of the insufficient supply of nutrients linked thereto the cells have shut down their metabolism, in which case the initially slightly basic nutrient medium generally by virtue of lactate elimination becomes slightly acid and slightly toxic, for example as a consequence of enrichment with ammonia from the breakdown of glutamine.

In addition the cells are energetically weakened in anaerobic glycolysis and the oxygen content in the medium moves back into the saturation range again. In that situation an excess supply of oxygen can even suddenly occur, which in the absence of nutrients is also no longer required or used. That damages the cells because of increased immune deficiency by virtue of radical catcher molecules which are scarcely still present, in relation to oxygen radicals which are constantly re-forming. When the nutrient medium is used it is sucked away and the cells coated with newly fresh nutrient medium. The superfluity-hunger cycle begins afresh.

In static cultures, by virtue of that cyclic culture non-homogeneity, it is scarcely possible to carry out reliably reproducible experiments with uniform validity. For example the timing is of extreme significance for the delivery of medicaments. If the medicaments are supplied at the beginning of the feed cycle the cells are in a good energy state and highly metabolically active. They accordingly react entirely differently as soon as the metabolism switches over to the hunger state which subsequently becomes increasingly worse. In summary it can be found that cultivated cells in the classic batch method do not involve homogeneous culture conditions at any time in the culture period. It is correspondingly difficult to achieve significant test results in relation to medicament effects in vitro. Nonetheless static cultures are frequently used for experiments as this involves a simple system entailing low costs and high sample throughput.

To overcome the disadvantages of static methods, so-called perfusion culture methods have become known in the state of the art, which have a number of scientifically proven advantages over the classic method of cultivating cells. For example, similarly to the situation in the body, care is taken to ensure that the cultured cells are exposed to uniform environmental conditions over the entire culture period. Conventional perfusion devices have special perfusion chambers which are slightly overpressure-tight and in which the cells are cultivated. Nutrient medium is fed from a nutrient medium reservoir to the perfusion chamber by way of a hose system and a pump. The reservoir is generally brought to body temperature and gassed with an air-CO₂ mixture, similarly to the incubator. In that way care is taken to ensure that the cells are fed continuously fresh medium as required with a regulatable flow rate through the perfusion chamber. Constant environmental conditions thus obtain over the entire culture period. That in turn ensures meaningful and reproducible test results which can be achieved very substantially independently of the administration timing of test substances.

U.S. Pat. No. 8,501,462 discloses a perfusion device having a multi-well plate in which there are provided a plurality of inserts which each have a perfusion region. Arranged at the underside of the inserts is a gas-permeable membrane, wherein the membrane at the side towards the respective insert has projections, by means of which the membrane is spaced from the underside of the insert. The intermediate space between the projections forms a flow passage, through which the nutrient can flow, and in so doing diffuses by way of the membrane to the cell cultures. A disadvantage with that configuration is that the nutrient medium flows for the major part directly from the feed line to the discharge line. In addition at the polygonal edges of the inserts this arrangement involves differing nutrient availability, by virtue of a different flow speed of the nutrient medium in the edge region and by virtue of the resulting turbulence there. For that reason there are large zonal differences in terms of availability of the nutrients, and they are detrimental for homogeneous growth of the cell cultures and therefore unwanted.

US No 2013/0068310 concerns a perfusion device of a multi-layer structure, having a multi-well plate. In that arrangement disposed in a distribution layer and in a gradient layer are micropassages serving to produce a concentration gradient of nutrient medium above the cell culture. The nutrient medium is delivered by way of a porous membrane. Various geometrical arrangements are provided for the micropassages of the gradient and distribution layers, wherein the micropassages are connected by way of a network to connecting passages. That results in a field-like concentration gradient of nutrient medium, wherein this involves high flow compression of the nutrient medium at the direction-changing points and a constant flow density occurs only in partial regions. It is therefore also not possible with this diffusion device to provide homogeneous culture conditions for the cell cultures by virtue of detrimental nutrient supply. That is extremely undesirable for test series with verifiable results.

A disadvantage with the perfusion devices known in the state of the art is in particular their complexity. Examples of such complex perfusion devices are disclosed in WO 82/03227, WO 02/24861, WO 2015/027998, U.S. Pat. No. 6,670,170, DE 4305405, DE 19742163 and DE 10118905. The consequence of this is that one-way systems for perfusion cultures are scarcely obtainable and if so then only with such small growth surfaces for the cell cultures that only special individual investigations on cells are meaningful and possible. In the case of multi-way systems in turn operation and here in particular sterile assembly require a great deal skill by virtue of the complex devices, and that usually results in a low level of sample throughput.

WO 2007/124481 and WO 2011/011350 concern microfluid devices, wherein nutrient and test substances are fed to individual cells by way of micropassages. In part the flow of the substances is produced exclusively by the capillary forces acting in the closed micropassages. In that case the investigation objects are either incorporated into the wall of the micropassage or are in holders specifically arranged for that purpose. As investigations can only be carried out on individual objects with those devices the microfluid devices are of a closed structure and have an integrated perfusion device which cannot be replaced. In particular only dynamic but not static cell cultures can be investigated with those devices. In addition the investigation objects are in part flushed out of the microfluid device by virtue of the configuration of the micropassages, whereby homogeneous culture conditions for cell cultures are also made impossible.

To sum up the perfusion culture method with the perfusion devices known in the state of the art is technically much more complicated and thus more expensive to operate than the conventional batch method, wherein in particular the sterile assembly and the more time-consuming handling of the existing devices has resulted in a lower level of acceptance of this alternative which in itself is physiologically more expedient, to classic in vitro models.

Therefore the object of the invention is to provide a perfusion device with which the disadvantages of the known devices for the perfusion culture method are avoided. In addition the invention seeks to provide a possible way of converting existing cell culture containers for static cultures easily into a device with which static cultures can be converted into perfusion cultures and cultivated with the perfusion culture method. In that respect the aim is to provide homogeneous culture conditions for the cell cultures, wherein the perfusion device can be inserted into the cell culture container without damaging the cells of the cell culture.

### SUMMARY OF THE INVENTION

With the perfusion device according to the invention static cell cultures (so-called “batch cultures”, “staple culture dishes” or “staple culture plates”) can be upgraded or converted to so-called “perfusion cultures” in the shortest time. Conversion back into a static cell culture is also quite easily possible. For that purpose, the perfusion device according to the invention has a perfusion region which is connected by way of supply line to at least one inlet opening and by way of a discharge line to at least one outlet opening. If the perfusion device has a plurality of inlet openings and/or a plurality of outlet openings a plurality of supply lines and discharge lines can be provided, which serve to connect the inlet openings and/or the outlet openings to the perfusion region.

The at least one inlet opening serves for the supply of nutrient medium for the cell cultures to be investigated, nutrient medium which has not been used and possibly excretion substances of the cell cultures are discharged by way of the at least one outlet opening.

The perfusion device has a perfusion region, by way of which nutrient medium is delivered to the cell cultures to be investigated, the cell cultures being arranged in a cell culture container into which the perfusion region can be fitted. The term cell culture container in accordance with the invention is used in this respect to denote conventional dish-form containers which are known in the state of the art like for example petri dishes and at least partly plate-shaped containers like for example multi-well plates and other containers, which are used for the cultivation of microorganisms and for cell culture, in particular cell culture containers with a removable cover.

In that respect the cells, as is generally usual for static cell cultures, can firstly be seeded in the cell culture container and covered with nutrient medium. As soon as the cells are attached to the dish or plate bottom of the cell culture container static culture can be converted into a perfusion culture by the perfusion region of the perfusion device according to the invention being fitted into the cell culture container.

The inlet opening can be connected to a nutrient medium reservoir. The nutrient medium is fed by way of the supply line to the perfusion region, by way of which it is made available to the cell cultures in the cell culture container. In the case of a plurality of inlet openings nutrient medium can be supplied to the perfusion region by way of a plurality of supply lines. If the supply lines end at different locations in the perfusion region then particularly homogeneous delivery of the nutrient medium can be achieved, depending on the respective distribution of the supply lines. With a plurality of inlet openings it is also possible to supply nutrient medium by way of one inlet opening while nutrient media mixed with medicaments or other test substances are supplied by way of another inlet opening. In that way tests can be particularly easily carried out.

The perfusion region has a geometrical structure, by way of which the nutrient medium is made available to the cell cultures. In the inserted state of the perfusion region the structure is towards the cell cultures. The structure provides a passage which connects the supply line and the discharge line and through which the nutrient medium can flow. In that case the geometrical structure is of such a configuration that the passage formed covers large parts of the perfusion region. In that way it is possible on the one hand for regions of large area to be made available, through which the nutrient medium flows. Accordingly regions of large area are also made available with nutrient medium for the cell cultures in the cell culture container. On the other hand the passage causes a flow of the nutrient medium at a certain flow rate so that the disadvantages of static cell culture methods do not arise, but in return there are the advantages of perfusion culture methods.

The passage can be in the form of a groove or channel, wherein—depending on the respective geometrical structure—variable groove depths and groove widths but also at least partially or entirely constant groove depths and/or groove widths are possible. Insofar as the geometrical structure in the inserted condition is spaced from the bottom of the cell culture container it is possible to insert the perfusion device without damaging or indeed destroying the cell cultures. In that way static cell cultures can be converted into perfusion cultures without adversely affecting the cells. The spacing of the outermost region of the geometrical structure relative to the bottom of the cell culture container in the inserted state of the perfusion region corresponds in that case to the spacing of the passage or the geometrical structure relative to the bottom of the cell culture container.

It is possible by means of the invention to use the petri dishes employed for conventional cell culture methods, but also other cell culture containers like for example multi-well plates, for perfusion methods, by using a perfusion device according to the invention, whose perfusion region is fitted into the cell culture container, instead of the conventionally employed cover. The classic formats of cell culture containers can thus be retained. By removal of the perfusion device the system can be converted from a perfusion culture back into a conventional static culture. The perfusion device according to the invention is very simple to handle and there is no disadvantage in respect of time for a perfusion culture method operated therewith in comparison with the batch method.

In an advantageous embodiment the perfusion device is in the form of a cover for the cell culture container. In that case the normal cover, for example the normal petri dish cover or the normal covering of the multi-well plate can be removed and replaced by the perfusion device according to the invention. In that case the entire perfusion device can be integrated into the cover which is easy to change. Conversion of a static cell culture into a perfusion culture is therefore easily possible by replacing the cover of the cell culture container. Preferably it is further provided that the perfusion device has a holding element, with which the perfusion device is removable from the cell culture container. Replacement of the cover of the cell culture container is thus possible with a single handhold.

Preferably, the passage and/or the geometrical structure of the perfusion region in the inserted state is or are arranged substantially parallel to the bottom of the cell culture container. In that way, it is possible to provide that the nutrient medium flowing through the passage is at a substantially constant spacing relative to the cell cultures and thus homogeneous environmental conditions are made available for the cell cultures. In that respect in an embodiment it is provided that the geometrical structure in the inserted state is at a spacing of less than 250 μm relative to the bottom of the cell culture container, preferably a spacing of between 20 μm and 100 μm being provided.

In a further embodiment the perfusion region has a seal for sealingly closing off the cell culture container. For example the seal is arranged at the edge of the perfusion region and in the inserted state seals off the cell culture container, for example at the peripheral surface of a petri dish. It is possible in that way with simple means to achieve the overpressure-tight conditions as in a conventional complicated perfusion chamber.

In an embodiment the perfusion device has a cover plate on which a holding element can be arranged for removal thereof. The perfusion region can be arranged spaced from the cover plate. If the perfusion region itself is plate-shaped it can be provided that the perfusion region and the cover plate are arranged parallel. By virtue of a spacing the perfusion region can also be used in relation to cell culture containers in which the growth surface for the cell cultures is at a greater spacing relative to the upper edge of the container. In addition, with such perfusion regions, it is particularly easy to provide for sealing closure to the cell culture container.

The perfusion region itself can be at least partially or also entirely of a circular or cylindrical configuration. Such perfusion regions are inserted into circular petri dishes with cylindrical peripheral surfaces or for example into multi-well plates having cylindrical chambers for the cell cultures. It will be noted however that with cell culture containers of different shapes, for example square containers, it is also possible for the perfusion region to be of a shape adapted to the cell culture container and to be for example rectangular.

It is preferably provided that the passage formed by the geometrical structure is continuous and for example in the form of a continuous groove or channel connects the supply line to the discharge line. In that respect it can be provided that the end of the supply line, disposed in the perfusion region, and/or the end of the discharge line, disposed in the perfusion region, opens directly into the passage. With a continuous passage it is more easily possible to provide for a complete circulation of the nutrient medium.

The passage is at least partially of a spiral shape, wherein in an embodiment the passage is entirely of a spiral configuration. In that way it is possible to cover large regions of the perfusion device, wherein uniform nutrient supply and thus homogeneous culture conditions are afforded. The supply line can open into an end of the spiral-shaped part while the discharge line connects at the other end of the spiral-shaped part. Particularly in the case of circular perfusion regions a large part of the surface of the perfusion region can be covered by a spiral passage. If the growth surface of the cell cultures in the cell culture container substantially corresponds to the area of the perfusion region that means that a large region of the growth surface is covered by the nutrient medium flowing through the passage. It can be provided that the passage is entirely of a spiral configuration. It is however also possible for only a certain part of the passage to be of a spiral configuration.

In a preferred embodiment the geometrical structure is in the form of a web (in other words: a channel wall) arranged on the perfusion region. In that case the passage can extend between the web or in the form of a space region which is afforded between the web. The spacing of the outermost region of the web relative to the bottom of the cell culture container in the inserted state of the perfusion region corresponds in that case to the spacing relative to the bottom of the cell culture container.

In a preferred embodiment the web has curved side flanks. The side flanks of the web can be connected in that case by way of a flat connecting region or however they can converge to a more or less sharp edge. A curved transition without sharp edges is also possible. Oxygen gradients which represent a problem in particular in static cell culture methods can be avoided by the curved side flanks.

The passage can be in the form of a groove or channel between the geometrical structure in the form of the web. The web forms the wall of the passage. With curved side flanks that can afford a passage which is of curved cross-section, in particular a semicircular passage. Other geometries like for example partly elliptical or passages which are oval in some other way are however also possible. In particular there can be a varying curvature. At its outermost region it can be provided that the web is also curved, for example curved outwardly, so that the transition between two grooves does not have any sharp edges to avoid gradient formation.

In a preferred embodiment a passage is downwardly open. In that way the nutrient medium can pass directly and unimpededly to the cell structures, whereby once again gradient formation can be avoided.

In an embodiment of the invention the cross-sectional area of the passage is between 2.5 mm² and 20 mm², preferably between 8 mm² and 16 mm². The height of the geometrical structure or the height of the web is between 0.5 mm and 5 mm, preferably between 2 mm and 2.5 mm. The term height of the geometrical structure or the height of the web is intended in that respect to denote the depth of the passage. That is that dimension which in the inserted state of the perfusion device is arranged perpendicularly to the bottom of the cell culture container and is measured from the uppermost point of the web to the deepest point of the groove.

The passage formed by the geometrical structure permits a flow speed for the nutrient medium. The passage can be of such a configuration as to permit media flows of up to 10 ml/min, wherein media flows of between 0.2 ml/h and 1 ml/h are preferred to avoid cell-damaging shearing forces. The passage permits homogeneous perfusion over the entire growth surface without gradient zoning. That occurs in particular when using a curved passage shape.

The geometrical structure can be of such a configuration that the nutrient medium diffuses between the grooves. That can avoid a build-up of pressure in the system while microcirculation and circulatory flow of the nutrient medium is promoted.

For the same purpose the web and/or the passage can have devices serving for flow deflection for the nutrient medium flowing therethrough. This can involve small barriers or chicanes. The flow deflection devices also improve microcirculation and circulatory flow of the nutrient medium in the passage. In addition the flow transfer of unused nutrient medium is optimised so that this affords improved gas and nutrient substance availability for the cultivated cells.

The inlet opening and/or the outlet opening can be connected to a pump with which the flow of the nutrient medium is produced and fresh nutrient medium is supplied and used nutrient medium is discharged. Possibilities here are for example perfusion pumps, peristaltic pumps, injection pumps and the like.

A further embodiment provides that the perfusion device has a first and a second inlet opening for the supply of nutrient medium and a first and a second outlet opening for the discharge of as yet unused nutrient medium. In that case for example nutrient medium can be supplied in the first inlet opening while medicaments or other test substances are supplied in the second inlet opening. It is however also possible that exclusively nutrient medium or nutrient medium already mixed with medicaments or test substances is supplied both with the first and also with the second inlet opening. In that case the perfusion region is connected to the first inlet opening by way of a first supply line and to the second inlet opening by way of a second supply line. It is however also conceivable for the first and second inlet openings to open into the same supply line. The perfusion region is connected to the first outlet opening by way of a first discharge line and to the second outlet opening by way of a second discharge line. It is however also conceivable for both the first and also the second outlet openings to open into a discharge line. In addition is basically also possible to provide more than two inlet openings and more than two outlet openings. In addition the number of inlet openings does not have to coincide with the number of outlet openings.

By the provision of a plurality of inlet openings it is possible to still more homogenise the supply of the nutrient medium, for example by the supply lines being suitably distributed on the perfusion region. In addition, zonal investigations can be carried out by a plurality of inlet openings, by different nutrients or different medicaments or other test substances being supplied by way of the individual openings, then being passed to the perfusion region by way of the corresponding supply lines and made available there to the cell cultures. Different cell culture reactions then occur in the corresponding zones.

With at least two inlet and two outlet openings it is preferably provided that the geometrical structure connects the first supply line to the first discharge line in the form of a first passage and the second supply line to the second discharge line in the form of a second passage. In that respect it is preferably provided that the first passage and the second passage have the nutrient medium flowing therethrough in opposite relationship. The circulatory flow of the nutrient medium and the microcirculation are further improved by the opposite flow.

To be able to cover large-area regions of the perfusion region it can be provided that the first and second at least partially spiral-shaped passage are at least entirely of a spiral shape, with oppositely directed spirals being preferred.

The invention further concerns a set including a perfusion device as described above and a cell culture container, in particular a petri dish.

In the case of a cell culture container having a plurality of chambers for the cultivation of cells, in particular in the form of a multi-well plate, it can be provided that the perfusion device has a plurality of perfusion regions, wherein a respective perfusion region can be inserted in a chamber.

### BRIEF DESCRIPTION OF THE DRAWINGS

Further details and advantages of the present invention will be described more fully hereinafter by means of the specific description with reference to the drawings in which:

FIG. 1 is a diagrammatic view of a device for static cell culture according to the state of the art,

FIGS. 2a-2c are a perspective view, a side view, and a plan view from below of a perfusion device according to the invention,

FIGS. 3a and 3b are two cross-sectional views of the perfusion device according to the invention,

FIGS. 4a-4g are a perspective view, two side views, a plan view from below, a plan view from above, and two cross-sectional views of a further embodiment of a perfusion device according to the invention,

FIGS. 5a-5g are a perspective view, two side views, a plan view from below, a plan view from above and two cross-sectional views of a further embodiment of a perfusion device according to the invention,

FIGS. 6a-6h are a perspective view, two side views, a plan view from below, a plan view from above and two cross-sectional views of a further embodiment of a perfusion device according to the invention,

FIGS. 7a-7c are diagrammatic views of devices for flow deflection for the nutrient medium, and

FIGS. 8a and 8b are diagrammatic views of two sets according to the invention.

### DETAILED DESCRIPTION OF THE INVENTION

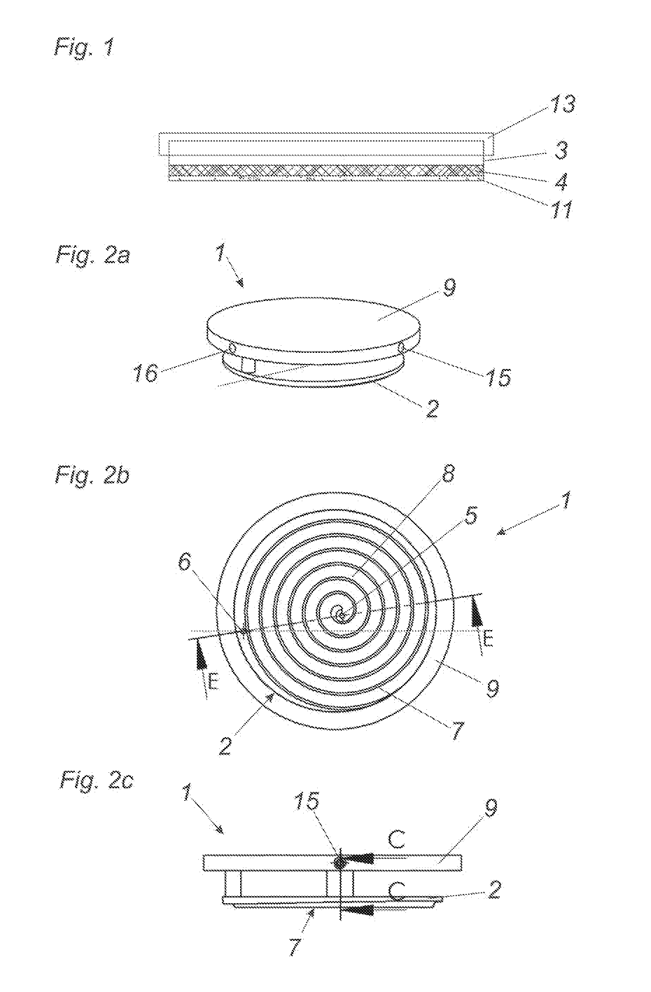

FIG. 1 shows a device for static cell culture, wherein cells 11 are arranged at the bottom of a cell culture container 3 in the form of a petri dish and are coated over with nutrient medium 4. The cell culture container 3 is closed with a cover 13.

The cover 13 can be replaced by a perfusion device 1 according to the invention whereby the existing cell culture container 3 together with cells 11 already cultivated therein can be used for the perfusion culture method.

The perspective view in FIG. 2a shows a perfusion device 1 according to the invention having a cover plate 9 and a perfusion region 2 spaced therefrom. The perfusion region 2 like the cover plate 9 is of a round circular configuration and can be inserted for example in a cell culture container 3 in the form of a circular petri dish. The Figure further shows the inlet opening 15 by way of which nutrient medium 4 can be supplied and the outlet opening 16 by way of which unused nutrient medium or the excretion substances of the cells 11 can be discharged.

FIG. 2b shows a plan view from below of the perfusion device 1. Shown in the middle of the circular perfusion region 2 is the supply line 5, by way of which the perfusion region 2 is connected to the inlet opening 15. The supply line 5 opens into the spiral passage 8 formed by the geometrical structure 7 in the form of a spiral web. Large regions of the perfusion region 2 are covered by the spiral shape of the passage 8 and can have the nutrient medium 4 flowing therethrough. The perfusion region 2 with the geometrical structure 7 arranged thereon represents a perfusion screw spiral, wherein the passage 8 is formed by the grooves of the screw spiral.

In this embodiment the supply line 5 and the discharge line 6 are at least partially in the form of tube connections, the tubes permitting spacing of the perfusion region 2 from the cover plate 9.

FIG. 2c shows a side view of the perfusion device 1 according to the invention, at which it is possible to clearly see the spacing between the cover plate 9 and the also plate-shaped perfusion region 2 as well as the geometrical structure 7 arranged on the perfusion region 2. The inlet opening 15 is also shown.

The perfusion device 1 according to the invention can be designed as a sterile one-way system and produced for example using an injection moulding process. The material involved is preferably plastics like for example polystyrene, PE plastics, PP plastics, PET plastics, PTFE plastics and the like.

After the perfusion region 2 is inserted into the cell culture container 3 items of equipment which are known per se for the perfusion method in the state of the art can be connected to operate a perfusion culture method. The inlet opening 15 or the outlet opening 16 can be connected to a pump, preferably a multi-channel perfusion pump, for which purpose hoses, preferably gas-impermeable hoses, like for example Neoprene, and connecting adaptors, preferably screwable Luer one-way connectors, can be used. A nutrient medium reservoir, for example a waterbath or an incubator, serves for supplying the cell culture with nutrient medium 4 which is optionally additionally gassed.

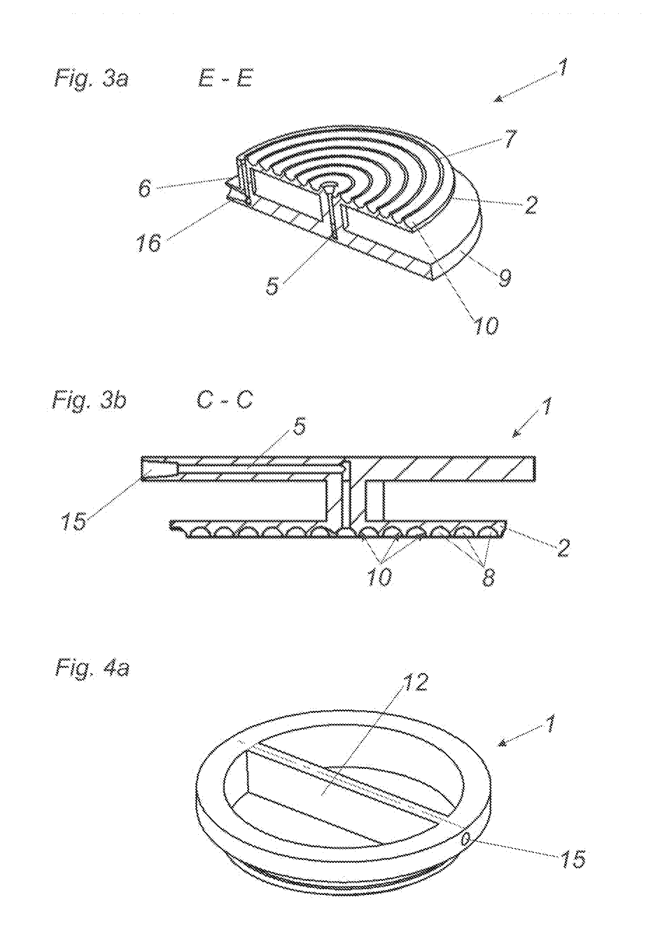

FIG. 3a shows a cross-sectional view of the perfusion device 1 along the section line EE shown in FIG. 2b. It can be seen that the geometrical structure 7 which is in the form of a web has curved side flanks 10 so that the passage 8 is of a semicircular cross-section. It is further possible to see the supply line 5 which connects the inlet opening 15 to the perfusion region 2 and in that case opens into the spiral passage 8 as well as the discharge line 6 which is arranged at the outer edge of the spiral passage 8 and connects the outlet opening 16 to the perfusion region 2.

FIG. 3b shows a cross-sectional view along section line CC in FIG. 2c. Once again it is possible to see the semicircular cross-section of the passage 8 which is formed by curved side flanks 10 of the geometrical structure 7 in the form of a web. It is possible to see the supply line 5 which is in the form of a tube connection and which connects the inlet opening 15 to the perfusion region 2.

Alternatively the inlet opening 15 and the outlet opening 16 can also be interchanged so that the fresh nutrient medium 4 is supplied at the outer edge of the spiral passage 8.

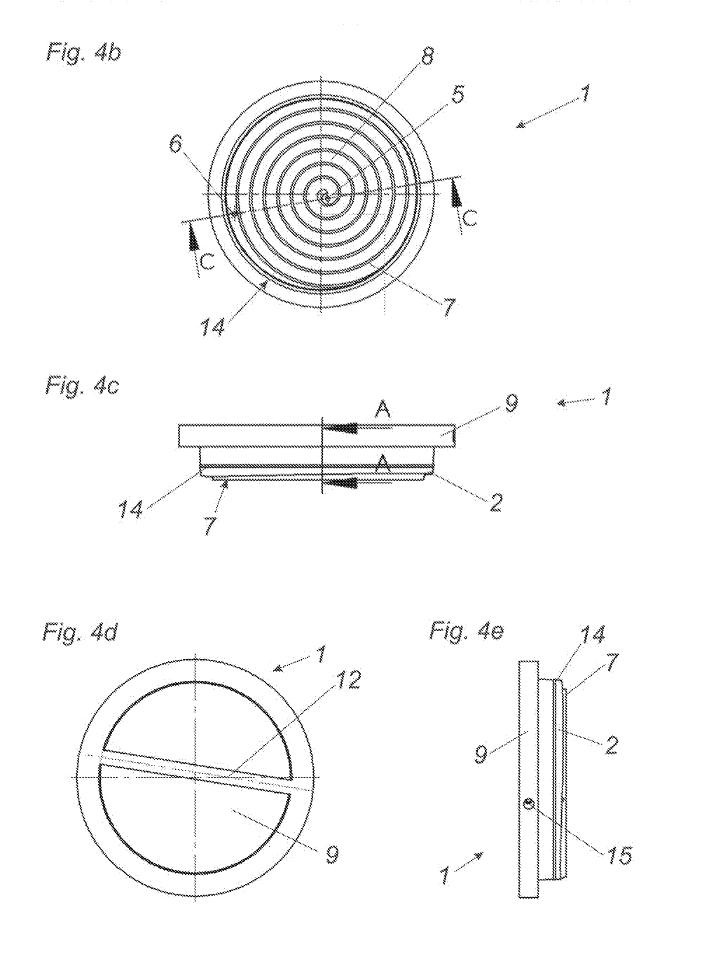

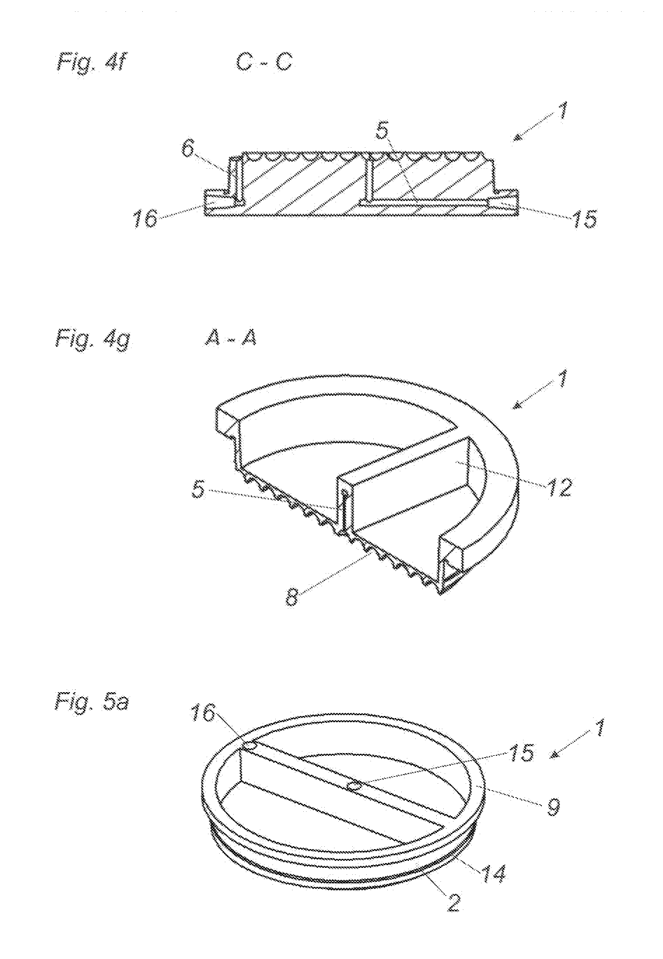

FIGS. 4a to 4f show a further embodiment of the perfusion device according to the invention, which substantially differs from the perfusion device 1 of FIGS. 2a to 2d and 3a and 3b in that the cover plate 9 has a holding element 12 with which the perfusion device 1 together with perfusion region 2 can be inserted with one handhold into the cell culture container 3.

The side views in FIGS. 4c and 4e show a seal 14 which is arranged around the edge of the perfusion region 2 and serves for sealingly closing off a circular cell culture container 3, for example a petri dish. FIG. 4b shows a plan view of this embodiment of the perfusion device 1 from below, substantially corresponding to the view in FIG. 2b. FIG. 4b shows a plan view from above, showing the holding element 12.

FIG. 4f shows a cross-sectional view along section line CC in FIG. 4b. It is possible to see the supply line 5 which in this embodiment is in the form of a passage and the discharge line 6 which is in the form of a passage and with which the inlet opening 15 and the outlet opening 16 are connected to the perfusion region 2.

FIG. 4g shows a sectional view along section line AA in FIG. 4c, in which it is possible to see once again the supply line 5 and the semicircular cross-section of the passage 8 which is formed by the curved side flanks 10 of the geometrical structure 7 in the form of the web.

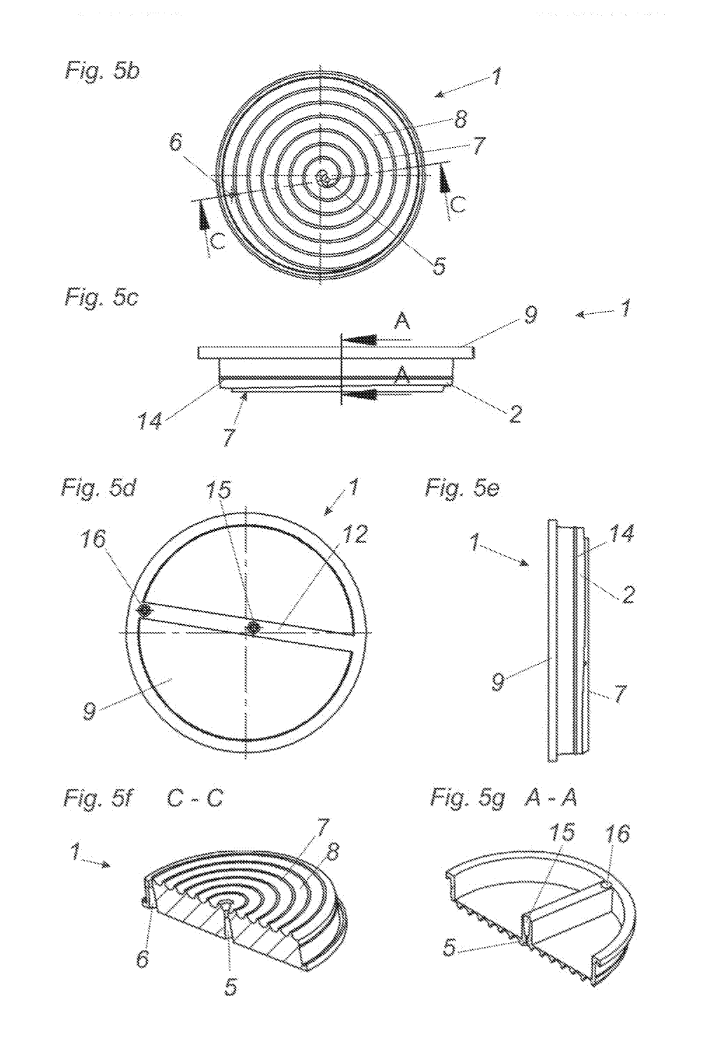

The embodiment of the perfusion device 1 according to the invention shown in FIGS. 5a to 5g differs from the embodiment shown in FIGS. 4a to 4g substantially only in that both the inlet opening 15 and also the outlet opening 16 are arranged on the top side of the cover plate 9 and not on the side surface.

As shown by means of the cross-sectional view shown in FIG. 5f along section line CC in FIG. 5b this arrangement of the inlet opening 15 and the outlet opening 16 involves the advantage that the supply line 5 and the discharge line 6 can be less complicated and can be implemented with shorter portions which in this embodiment are in passage form. That is also shown in the cross-sectional view in FIG. 5g showing a cross-section taken along section line AA in FIG. 5c. This view shows the supply line 5 which goes directly into the inlet opening 15. Both the inlet opening 15 and also the outlet opening 16 are arranged directly above the mouth opening of the supply line 5 and the discharge line 6 connecting to the perfusion region 2.

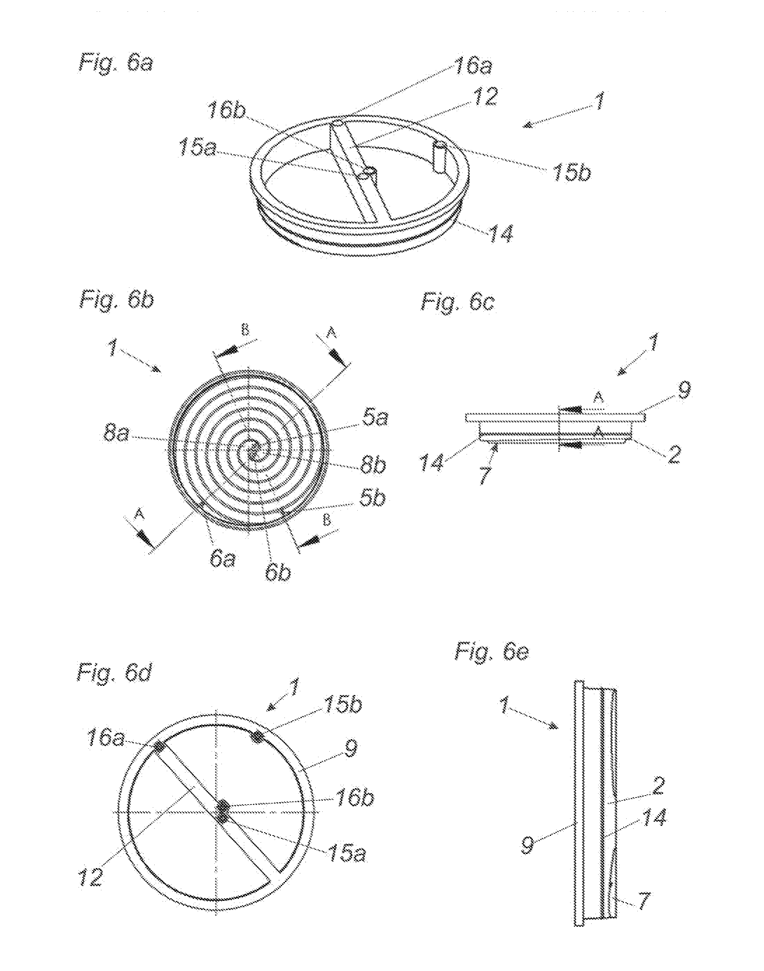

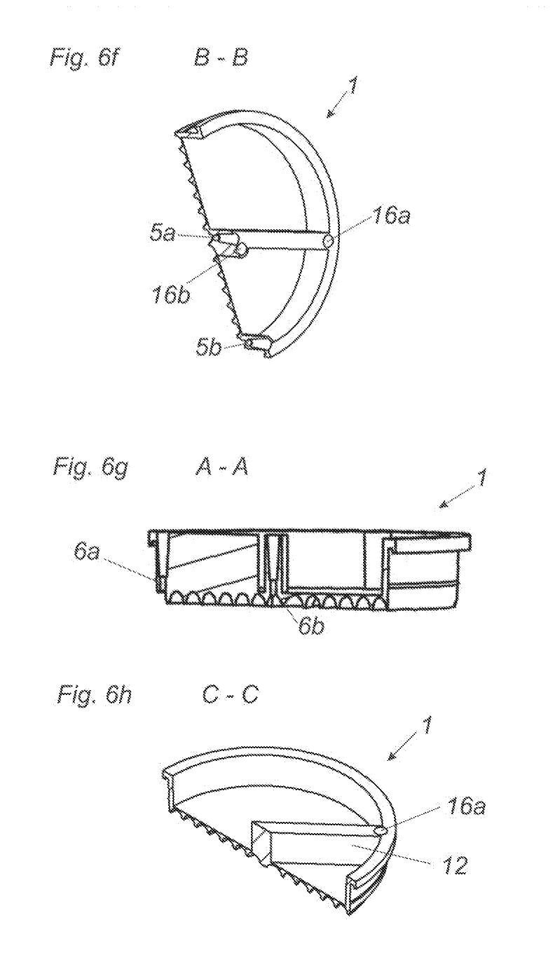

FIGS. 6a to 6h show a further embodiment of the perfusion device 1 according to the invention. Once again the perfusion device 1 has a round cover plate 9 with a holding element 12 for easily inserting the perfusion region 2, which is also round, in a cell culture container 3. The perfusion region 2 is edged around by a seal 14 serving for sealingly closing off the cell culture container 3.

In this embodiment the perfusion device 1 has a first inlet opening 15a arranged centrally in the cover plate 9. A second inlet opening 15b is arranged at the edge of the cover plate 9. The first inlet opening 15a and the second inlet opening 15b are connected to the perfusion region 2 by way of a first supply line 5a and a second supply line 5b. The first supply line 5a and the second supply line 5b are particularly clearly shown in the cross-sectional view of FIG. 6f illustrating a cross-section along section line BB in FIG. 6b. As the first inlet opening 15a and the second inlet opening 15b are arranged directly above on the top side of the cover plate 9 the first supply line 5a and the second supply line 5b can be kept short.

A first outlet opening 16a is arranged at the edge of the cover plate while a second outlet opening 16b is arranged centrally in the proximity of the first inlet opening 15a. The first outlet opening 16a and the second outlet opening 16b are connected to the perfusion region 2 by way of a first discharge line 6a and a second discharge line 6b. That can be clearly seen in the cross-sectional view in FIG. 6g illustrating a cross-section along section line AA in FIG. 6b. Once again the first discharge line 6a and the second discharge line 6b can be kept short as the first outlet opening 16a and the second outlet opening 16b are arranged directly above at the top side of the cover plate 9.

As can be seen from the view from below in FIG. 6b the first supply line 5a opens into a first passage 8a into which the first discharge line 6a also opens. The second supply line 5b like the second discharge line 6b opens into a second passage 8b. Both the first passage 8a and also the second passage 8b are of a spiral-shaped configuration. The first passage 8a and the second passage 8b are formed by the intermediate spaces of a spiral-shaped web representing the geometrical structure 7 of the perfusion region 2.

In general the nutrient medium 4 and/or a test substance or medicaments pass into the passage 8 at the supply line 2 and flow through the passage in the direction of the discharge line 6 where the unused nutrient medium 4 is discharged optionally jointly with excretion products from the cells 11.

In the present case the nutrient medium 4 passes into the first passage 8a at the first supply line 5a and flows through same in the direction of the first discharge line 6a where it issues from the first passage 8a. The nutrient medium 4 and/or a test substance or medicaments pass into the second passage 8b at the second supply line 5b and flow through same in the direction of the second discharge line 6b where the unused nutrient medium issues from the perfusion region 2 optionally with excretion products from the cells 11. As in this embodiment the first supply line 5a is arranged centrally while the second supply line 5b is arranged at the outer edge the nutrient medium 4 flows through the first passage 8a and the second passage 8b in opposite relationship.

If for example along the passage 8a and 8b a total of 20% of the supplied nutrient medium 4 is used, that is to say 80% of the supplied fresh nutrient medium 4 issues again at the outlet opening, it is then possible by virtue of the oppositely directed flow to provide on average around 90% of fresh nutrient medium 4 for the cells 11. Accordingly extremely homogeneous environmental conditions obtain for the cells 11, which has been found to be extremely advantageous for the investigations into the behaviour of the cells 11, for example with the supply of certain medicaments or test substances. The oppositely directed flow relationship of the first spiral-shaped passage 8a and the second spiral-shaped passage 8b can be particularly clearly seen in FIG. 6b.

FIG. 6h shows a cross-sectional view along section line CC in FIG. 6c.

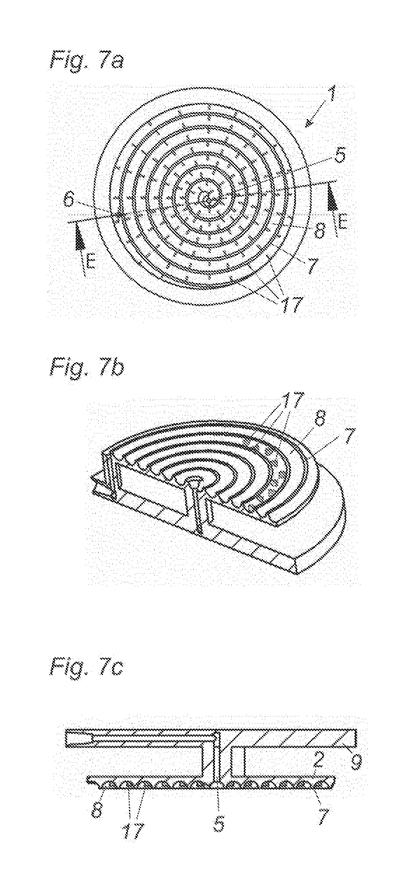

FIG. 7a shows a plan view of a further embodiment of a perfusion device 1 according to the invention with a geometrical structure 7 which is in the form of a spiral-shaped web and the intermediate spaces of which form an also spiral-shaped passage 8 through which the nutrient medium 4 can flow, the nutrient medium 4 entering at a supply line 5 which opens into the passage 8 and issuing at a discharge line 6 which also connects to the passage 8. Arranged on both sides of the web are devices 17 for flow deflection in the form of chicanes which lead to microcirculation and better circulatory flow of the nutrient medium 4 flowing therethrough.

FIG. 7b shows a diagrammatic cross-sectional view showing the arrangement of the flow deflection devices 17 arranged in the side flanks 10 of the web, with which the microcirculation of the nutrient medium 4 flowing therethrough is increased.

FIG. 7c shows a cross-sectional view along section line EE in FIG. 7a, also showing the flow deflection devices 17 which are arranged as chicanes at the side flanks 10 of the geometrical structure 7 which is in the form of a web.

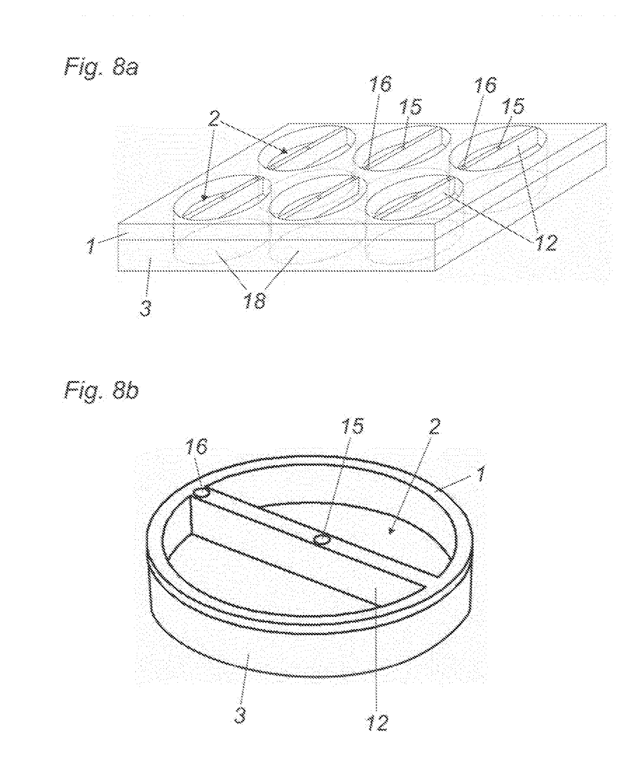

FIG. 8a shows a perspective view of a set according to the invention including a cell culture container 3 which is in the form of a multi-well plate and which has a plurality of chamber-shaped recesses 18 (shown in broken line), on the bottom of which cell cultures 11 are arranged. The perfusion device 1 can serve as a cover for the multi-well plate. The perfusion device 1 has a plurality of perfusion regions 2, each perfusion region 2 being inserted into one of the chamber-shaped recesses 18. In addition the perfusion device 1 has a plurality of holding elements 12 respectively associated with a perfusion region 2. In addition an inlet opening 15 and an outlet opening 16 is associated with each perfusion region 2. The inlet opening 15 and the outlet opening 16 are connected to the respective perfusion region 2 by way of a supply line 5 and a discharge line 6 so that each individual chamber-shaped recess 18 can be supplied with nutrient medium 4. As a respective inlet opening 15 and outlet opening 16 are associated with each chamber-shaped recess 18 the individual recesses 18 can be supplied with differing nutrient media so that a plurality of different tests can be carried out at the same time.

FIG. 8b shows a diagrammatic view of a set according to the invention comprising a cell culture container 3 in the form of a petri dish and a perfusion device 1, the perfusion region of which is inserted into the petri dish.

1. A perfusion device for a cell culture container, in particular a petri dish, comprising at least one inlet opening for the supply of nutrient medium and at least one outlet opening for the discharge of unused nutrient medium, wherein there is provided at least one perfusion region for the delivery of nutrient medium to cell cultures, which is connected to the inlet opening by way of a supply line and to the outlet opening by way of a discharge line, wherein the perfusion region can be inserted into the cell culture container, wherein the perfusion region comprises a geometrical structure which connects the supply line and the discharge line in the form of an at least partially spiral-shaped passage through which the nutrient medium can flow, wherein the geometrical structure is spaced from the bottom of the cell culture container in the inserted state.

2. The perfusion device according to claim 1, wherein the perfusion device is in the form of a cover for the cell culture container.

3. The perfusion device according to claim 1, wherein the passage connecting the supply line and the discharge line is entirely spiral-shaped.

4. The perfusion device according to claim 1, wherein the passage of the perfusion region or the geometrical structure or both, the passage and the geometrical structure in the inserted state is or are arranged parallel to the bottom of the cell culture container.

5. The perfusion device according to claim 4, wherein the geometrical structure in the inserted state is at a spacing of less than 250 μm, preferably between 20 μm and 100 μm, relative to the bottom of the cell culture container.

6. The perfusion device according to claim 2, wherein the perfusion region has a seal for sealingly closing off the cell culture container.

7. The perfusion device according to one claim 1, wherein the perfusion device has a cover plate and the perfusion region is arranged spaced from the cover plate.

8. The perfusion device according to claim 1, wherein the passage is of a continuous configuration.

9. The perfusion device according to claim 1, wherein the passage is downwardly open.

10. The perfusion device according to claim 1, wherein the geometrical structure is in the form of a web, which is arranged on the perfusion region, wherein the passage extends between the web.

11. The perfusion device according to claim 10, wherein the web has curved side flanks.

12. The perfusion device according to claim 10, wherein the passage has a cross-sectional area of between 2.5 mm² and 20 mm², preferably between 8 mm² and 16 mm².

13. The perfusion device according to claim 10, wherein the web has a height of between 0.5 mm and 5 mm, preferably between 2 mm and 2.5 mm.

14. The perfusion device according to claim 10, wherein the web or the passage or both, the web and the passage has or have devices for flow deflection for the through-flow of nutrient medium.

15. The perfusion device according to claim 1, wherein there are provided a first inlet opening and a second inlet opening for the supply of nutrient medium and a first outlet opening and a second outlet opening for the discharge of unused nutrient medium, wherein the perfusion region
is connected to the first inlet opening by way of a first supply line,
is connected to the second inlet opening by way of a second supply line,
is connected to the first outlet opening by way of a first discharge line, and
is connected to the second outlet opening by a second discharge line.

16. The perfusion device according to claim 15, wherein the geometrical structure connects the first supply line to the first discharge line in the form of a first, at least partially spiral-shaped passage and the second supply line to the second discharge line in the form of a second, at least partially spiral-shaped passage, and wherein the nutrient medium can flow through the first passage and the second passage.

17. The perfusion device according to claim 16, wherein the first passage or the second passage or both, the first passage and the second passage is or are entirely spiral-shaped.

18. The perfusion device according to claim 16, wherein the first passage and the second passage are in the form of oppositely directed spirals.

19. The perfusion device according to claim 1, wherein there is provided a holding element with which the perfusion device is removable from the cell culture container.

20. A set including a cell culture container, in particular a petri dish, and the perfusion device according to claim 1, wherein the perfusion region can be inserted into the cell culture container.

21. The set according to claim 20, wherein the cell culture container has a plurality of chambers for the cultivation of cells, wherein the perfusion device has a plurality of perfusion regions and wherein a respective perfusion region can be inserted into a chamber.
